(19) United States
(12) Patent Application Publication
(10) Pub. No.: US 2025/0022581 A1
(43) Pub. Date: Jan. 16, 2025

(54) Item of Intervention Information in an Image Recording Region

(71) Applicant: Siemens Healthineers AG, Forchheim (DE)

(72) Inventors: Julian Wohlers, Erlangen (DE); Axel Joachim Krafft, Hemhofen (DE)

(73) Assignee: Siemens Healthineers AG, Forchheim (DE)

(21) Appl. No.: 18/770,994

(22) Filed: Jul. 12, 2024

(30) Foreign Application Priority Data
Jul. 13, 2023 (DE) 10 2023 206 682.3

(51) Int. Cl. G16H 30/40; G16H 30/20

(52) U.S. Cl. CPC G16H 30/40; G16H 30/20

(57) ABSTRACT

A computer-implemented method for providing an item of intervention information in an image recording region of an imaging device, the computer-implemented method including: positioning the examination object in an examination position in the image recording region, capturing an item of depth information relating to the examination object, capturing the item of intervention information, providing the item of intervention information as a function of the item of depth information, and outputting the item of intervention information into the image recording region. The disclosure further relates to an imaging device that is designed to execute the computer-implemented method.

### BACKGROUND

Independent of the grammatical term usage, individuals with male, female, or other gender identities are included within the term.

Interventional procedures are often accompanied by imaging methods, which allow doctors performing the treatment to observe and/or monitor the progress of the procedure on a video screen with the aid of captured image data relating to the interior of a patient. Dedicated monitors are usually installed in an intervention environment, giving a real-time display of the image data that is captured by an imaging device. In addition to medical instruments such as e.g. a needle or a catheter, it is also possible to display further items of information such as e.g. colored markings, which represent the entry and destination points of a needle path. The navigation or guidance of the medical instrument can be simplified or managed more safely thereby.

Projection onto a projection screen or video screen is, however, awkward in terms of the workflow since the doctor performing the procedure or treatment must repeatedly divert their attention away from the patient in order to follow the medical instrument with the aid of the projection. Furthermore, image data that is represented on conventional video screens usually has a different scale. Further challenges are presented in particular by imaging devices that have enclosed image recording regions, e.g., magnetic resonance devices, computed tomography systems, and the like. When using such imaging devices, a patient is partly concealed by a patient tunnel which can hamper the access to the patient as well as the positioning of a monitor. In the case of magnetic resonance devices, it is moreover necessary to use MRT-compatible devices and components, thereby limiting options for real-time monitoring of the instrument.

### SUMMARY

An object of the disclosure is, therefore, to increase the efficiency and safety of interventional procedures, which are accompanied by imaging.

This object is inventively achieved by the subject matter of the independent claims. Advantageous aspect variants and appropriate developments are specified in the subclaims.

The inventive method is preferably performed in a completely computer-implemented manner. According to the inventive method, items of intervention information are provided in an image recording region of an imaging device. The inventive method comprises steps as follows.

In a step, an examination object is positioned in an examination position in the image recording region.

An examination object can be a human or animal patient or part of a human or animal body. However, an examination object can equally be any desired object, e.g., a foodstuff or an archaeological find.

An imaging device can be any device that is designed to capture image data relating to an examination object, in particular from a body region of a patient. An imaging device is preferably designed to record two-dimensional and/or three-dimensional image data, in particular time-dependent three-dimensional image data relating to the examination object. Examples of imaging devices include magnetic resonance devices, x-ray devices, computed tomography devices, single photon emission computed tomography systems, positron emission tomography systems, but also mammography devices, ultrasound devices, and the like. In a preferred aspect variant, the imaging device is embodied as a magnetic resonance device.

An image recording region can be characterized by a volume or a region from which image data can be recorded using the imaging device. In particular, the image recording region can be structurally enclosed by the imaging device. For example, the image recording region is designed to record the examination object or a part of the examination object during an imaging examination. In particular, the image recording region can represent a volume of the imaging device that is formed by a patient tunnel. It is, however, also conceivable for the image recording region to represent a surface of an examination object and/or a volume containing part of an examination object. The surface and/or the volume of the examination object can contain, in particular, a diagnostically or therapeutically relevant body region.

An examination position can be characterized by a predefined position of the examination object relative to the imaging device. In particular, the examination position can be characterized by a predefined location, orientation, and/or posture of a patient during the interventional procedure and/or the imaging examination. For example, the examination position can be a lateral position, a supine position, a prone position, a sitting position, or similar, of a patient.

The positioning of the examination object in the image recording region can take place manually, e.g., by supporting a patient, but can also be partly or entirely automatic. For example, the examination object can be positioned in the image recording region as appropriate for the application by means of a patient table of a patient support device of the imaging device.

In a further step of the inventive method, an item of depth information relating to the examination object is captured.

The item of depth information is preferably captured by means of a suitable sensor. The item of depth information can contain three-dimensional items of information about the examination object. For example, the item of depth information can contain optical data, optical image data, items of profile information and/or items of contour information relating to the examination object. The three-dimensional item of information can comprise in particular data relating to a three-dimensional surface geometry of at least a part or section of the examination object. It is conceivable for the examination position of the examination object to be determined and/or verified as a function of the item of depth information.

A suitable sensor can comprise, e.g., a camera, in particular, a plurality of two-dimensional cameras and/or at least one three-dimensional camera. It is equally conceivable for the sensor to have an optical distance meter or an array of optical distance meters, which are designed to capture a three-dimensional item of information relating to the examination object. The sensor can also be part of a receiving system of the imaging device. For example, the items of depth information for the examination object can be created by the imaging device as a function of an overview measurement. It is conceivable, in particular, for the items of depth information to be captured by means of a local coil of a magnetic resonance device. Further examples of suitable sensors include ultrasound sensors, radar sensors, LIDAR sensors, and the like. The sensor is preferably designed to transfer the items of depth information relating to the examination object by means of a signal connection to a computing unit of the imaging device.

In a step, an item of intervention information is captured.

An item of intervention information can be any item of information that is relevant to the performance of an interventional procedure. The item of intervention information preferably comprises at least one element from the following list: an intervention plan, a navigation plan of a medical instrument in the examination object, an item of instrument information, a representation of a medical instrument, in particular a medical instrument which is arranged in the examination object, a representation of an incision point, a representation an anatomical structure, a representation of a destination position, a representation of an ablation volume, a representation of a temperature distribution and/or a representation of a degree of correspondence between the navigation plan for the medical instrument in the examination object and a position and/or orientation of the medical instrument in the examination object.

The item of intervention information can also comprise items of information relating to the examination object, in particular, items of patient information, an item of information relating to the procedure that is to be performed, and/or an item of instrument information. It is further conceivable for the items of intervention information to comprise anatomical image data that is captured by means of the imaging device. In addition to this, the item of intervention information can also comprise an item of communication information. It is thereby advantageously possible to allow communication between a medical operative who is performing treatment at the imaging device and persons who are located outside the intervention/examination environment or remotely from the imaging device.

The item of intervention information can be captured or received from a data storage entity in a cloud, a network and/or a local data storage entity, for example. The item of intervention information can also be extracted from a digital patient file or a digital patient register. Furthermore, the item of intervention information can be extracted from a treatment plan and/or an intervention plan. The item of intervention information can also be retrieved or received from a medical instrument, in particular a computing unit and/or a control unit of the medical instrument. The item of intervention information can be captured automatically and/or input manually by means of a user interface.

In a further step, the item of intervention information is provided as a function of the item of depth information.

In a preferred aspect variant of the inventive method, the provision of the item of intervention information comprises adapting the item of intervention information to the examination position of the examination object.

The provision of the item of intervention information can include, in particular, modifying, scaling, and/or adjusting a size, an orientation, a position, a shape, and/or a distortion of a representation which is comprised in the item of intervention information and/or image data which is comprised in the item of intervention information. In particular, the provision of the item of intervention information can include adapting the item of intervention information to the posture of a patient, an orientation of the medical instrument, and/or a three-dimensional item of information relating to the examination object. For example, the representation that is comprised in the item of intervention information and/or the image data that is comprised in the item of intervention information can be adapted in such a way that a substantially distortion-free projection is possible on a curved or rounded surface of the examination object.

By means of adapting the item of intervention information to the examination position of the examination object as a function of the item of depth information, the item of intervention information can advantageously be adapted to a posture and/or surface geometry of the examination object. It is advantageously possible thereby to compensate for a distortion of an item of intervention information that is projected onto the examination object at a curved surface of the examination object. It is furthermore advantageously possible to avoid scaling effects, e.g., differences between a distance that has actually been covered by the medical instrument in the examination object and a representation that is comprised in the item of intervention information in the image recording region. It is equally conceivable for the item of intervention information to be advantageously adapted to output by means of a plurality of video screens or projectors, in particular, a projection from a plurality of angles. It is thereby possible to avoid or reduce distortion effects when outputting the item of intervention information into the image recording region.

In a further step of the inventive method, the item of intervention information is output into the image recording region.

The output of the item of intervention information can include projecting the item of intervention information onto a surface in the image recording region, in particular onto a surface of the examination object. For example, the item of intervention information can be projected onto a surface of a patient by means of a projector while the patient is situated in the examination position.

In a further aspect variant of the inventive method, the output of the item of intervention information takes place in the image recording region, onto the examination object and/or a wall of the imaging device.

The item of intervention information can be output on a video screen that is positioned within the image recording region and/or projected onto a surface that is situated within the image recording region. In particular, the item of intervention information can be projected onto a curved wall of the patient tunnel, which encloses at least part of the examination object when the examination object is positioned in the examination position in an appropriate manner for the application. It is, however, equally conceivable for the item of intervention information to be projected onto part of a patient table and/or a component of the imaging device (e.g., a local coil, patient support, etc.).

The output of the item of intervention information into the image recording region can be real or virtual, e.g., by means of projection (real) or virtually by means of artificially augmented reality, mixed reality, or a virtual environment.

Furthermore, the output of the item of intervention information can include storing the item of intervention information on a storage unit, such as, e.g., a local data storage entity, a data storage entity in a network, and/or a cloud-based data storage entity.

By means of the inventive method, an item of intervention information can be provided directly in the image recording region of an imaging device, in particular directly on an examination object. It is thereby possible advantageously to avoid or mitigate the need for a doctor performing the treatment to divert their attention away from the examination object during an interventional procedure.

Furthermore, an item of intervention information can advantageously be adapted by means of the inventive method to a non-planar projection surface within the image recording region in order to avoid distortion effects. In particular, scaling differences between the examination object and an item of intervention information that is output can be avoided by virtue of the inventive method. It is thereby possible advantageously to reduce any risk of a medical instrument being incorrectly positioned during the interventional procedure.

Furthermore, by means of the inventive method, it is possible to simplify or shorten a clinical workflow for an interventional procedure in a patient tunnel of an imaging device since a repeated transfer of the examination object into and out of the patient tunnel, e.g., for the purpose of marking an incision point, is no longer necessary.

In an aspect variant, the inventive method comprises the step:
capturing anatomical image data relating to the examination object by means of the imaging device,
wherein the provision of the item of intervention information takes place as a function of the item of depth information and the anatomical image data relating to the examination object.

The anatomical image data relating to the examination object can comprise a two-dimensional and/or a three-dimensional depiction or representation of an interior of the examination object, in particular, a patient. It is conceivable for the anatomical image data to comprise diagnostically and/or therapeutically relevant image data relating to a body region of a patient. In a preferred aspect variant, the anatomical image data comprises magnetic resonance images or computed tomography images of a body region of the patient that is relevant to the interventional procedure.

The provision of the item of intervention information can include, e.g., modifying, scaling, and/or adjusting a size, an orientation, a position, a shape, and/or a distortion of a representation which is comprised in the item of intervention information, and/or image data which is comprised in the item of intervention information, as a function of the anatomical image data. It is further conceivable for the provision of the item of intervention information to comprise registering a representation, which is comprised in the item of intervention information, and/or image data, which is comprised in the item of intervention information, with the anatomical image data.

In a further aspect variant, the inventive method comprises the step:
registering the anatomical image data relating to the examination object with the item of depth information, and providing combined image data based on the registered anatomical image data and the item of depth information,
wherein the item of intervention information is provided as a function of the combined image data.

The provision of the item of intervention information as a function of the combined image data can take place in a similar manner to a previously described aspect variant, in which the item of intervention information is provided as a function of the item of depth information or the anatomical image data.

According to an aspect variant described above, the item of depth information relating to the examination object comprises three-dimensional data, in particular, optical data or optical image data. Registration of the anatomical image data with the item of depth information can take place using any desired image registration method. Examples of such image registration methods include area-based and/or feature-based methods on the basis of a correlation function, a correspondence of checkpoints, a global and/or local transformation, pattern recognition, a radial basis function, a Fourier transformation, or similar. The image registration method can further be complemented by the deployment of optical and/or magnetic markers, the use of orientation points, and/or a geometric equivalence of positioning of the examination object relative to the imaging device. Furthermore, the registration of the anatomical image data with the item of depth information can take place with reference to a model, in particular a body model, of a patient.

By virtue of providing the item of intervention information as a function of the anatomical image data relating to the examination object and/or the combined image data, the item of intervention information can advantageously be output in the same context as the anatomical image data, thereby reducing any risk of incorrect positioning of the interventional instrument.

In a further aspect variant, the inventive method comprises the step:
creating a model of the examination object as a function of the item of depth information relating to the examination object,
wherein the provision of the item of intervention information includes registering the model with the item of depth information, the anatomical image data, and/or combined image data,
wherein the combined image data is based on a registration of the anatomical image data relating to the examination object with the item of depth information.

A model of the examination object can comprise a three-dimensional model, in particular a body model of a patient.

The creation of the model of the examination object can take place as a function of reference values and/or standardized proportions. It is conceivable for the creation of the model to take place with reference to three-dimensional items of information and/or characteristic dimensions which are determined as a function of the item of depth information. Furthermore, the creation of the model can also take place on the basis of items of information relating to the examination object, in particular, on the basis of items of patient information such as, e.g., size, age, sex, and/or weight. In an aspect variant, the creation of the model includes adapting model parameters as a function of the items of depth information. The model of the examination object can also be created by means of a biogeneric algorithm and/or a trained algorithm that receives the item of depth information as input data.

The model of the examination object can, according to an aspect variant described above, be registered with the item of depth information, the anatomical image data, and/or the combined image data.

By using a model of the examination object, it is possible to reduce demands on a spatial resolution of the item of depth information. It is thereby possible advantageously not only to reduce the burden associated with the capture of the item of depth information, but also to reduce demand on the sensor which is used to capture the item of depth information.

By means of the model of the examination object, it is also possible advantageously to mitigate or compensate for errors in the capture of the item of depth information, thereby advantageously increasing not only the resilience of the inventive method but also safety for both the doctor performing treatment and the patient.

In a preferred aspect variant, the inventive method comprises the step:
capturing a movement of the examination object,
wherein the provision of the item of intervention information takes place as a function of the movement.

A movement can include a voluntary or involuntary movement of the examination object. In particular, a movement can be characterized by the movement of an organ, a heartbeat, a respiratory state, a swallow, or similar.

The respiratory state of the examination object can be characterized by a state of inhalation, exhalation, or a transition phase between inhalation and exhalation of a patient. In an aspect variant, the respiratory state of the examination object is captured by means of a respiration detection sensor, in particular a dedicated respiration sensor, and/or image-based respiration detection (e.g., based on the optical data and/or the anatomical image data). It is conceivable for the image-based respiration detection to be effected by means of an image processing algorithm that receives the item of depth information and/or the anatomical image data as input data.

Other movements of the examination object, such as e.g. the heartbeat, can equally be captured by means of dedicated sensors. Such dedicated sensors can be embodied as optical sensors or ECG sensors, for example. In addition to this, a pilot tone signal or comparable signals can also be used for capture and/or synchronization with the movement of the examination object.

The provision of the item of intervention information preferably comprises modifying, scaling, and/or adjusting a size, an orientation, a position, a shape, and/or a distortion of a representation which is comprised in the item of intervention information and/or of image data which is comprised in the item of intervention information, as a function of the movement of the examination object.

In particular, the provision of the item of intervention information can, as a function of the movement of the examination object, comprise adapting the output item of intervention information as a function of the respiration state of the examination object.

By adapting, as a function of the movement of the examination object, the item of intervention information that is output, positional changes of (anatomical) structures within the examination object due to a movement can be taken into consideration when the item of intervention information is output. It is thereby possible advantageously to reduce any risk of a medical instrument being incorrectly positioned.

In a further aspect variant of the inventive method, the capture of the item of intervention information comprises capturing an item of instrument information,
wherein the provision of the item of intervention information takes place as a function of the item of instrument information and
wherein the output of the item of intervention information includes outputting the item of instrument information onto the examination object in the image recording region.

An item of instrument information can be, for example, an item of information relating to the medical instrument such as e.g. a geometry and/or a diameter of a needle. The item of instrument information can, however, also comprise an item of information relating to navigation of the medical instrument in the examination object, e.g., a navigation plan and/or an item of information relating to an incision point. It is equally conceivable for the item of instrument information to comprise a planned ablation volume that is determined or estimated before the procedure, for example. Furthermore, the item of instrument information can comprise an item of information relating to an effect of the medical instrument in a tissue of the examination object, e.g., a temperature increase, a temperature distribution, a force absorption, a shear force, or similar. Such items of information can be determined, e.g., by means of the imaging device itself and/or by means of a sensor that is integrated into the medical instrument (e.g., temperature sensor, pressure sensor, force sensor, etc.).

The item of instrument information can be captured automatically, e.g., as a function of an item of patient information that is stored on a storage unit. The item of patient information can also comprise, in addition to any desired item of information relating to the patient, an item of information relating to the procedure that is to be performed, in particular, a position of the incision point and/or the intervention plan. It is, however, equally conceivable for the item of instrument information to be captured as a function of a user input and/or a signal connection with the medical instrument.

The provision of the item of intervention information can include, e.g., providing a representation of the incision point, a destination, and/or the intervention or navigation plan in a correct position relative to the examination object. Furthermore, the provision of the item of intervention information can include providing a representation of a temperature distribution, a force distribution and/or a planned ablation volume in the examination object. As a function of the item of depth information relating to the examination object, it is possible, according to an aspect variant described above, to take into consideration, in particular, an examination position and/or a curvature of a surface of the examination object in this case.

The output of the item of intervention information can include projecting the incision point, a destination position and/or the navigation plan into the image recording region, in particular onto a surface of the examination object.

By means of the inventive method, relevant items of information for an interventional procedure can be output on a surface of the examination object, it being advantageously possible to take a curvature of the surface of the examination object and/or a posture of the examination object into consideration. Furthermore, the withdrawal of a patient from the patient tunnel of the imaging device in order to manually mark the incision point is not necessary. It is thereby possible not only to improve the efficiency and quality of the interventional procedure but also to increase the safety of a patient.

In a further aspect variant of the inventive method, the capture of the item of instrument information includes capturing an intervention plan,
wherein the provision of the item of intervention information includes registering the intervention plan with a model that is created as a function of the item of depth information relating to the examination object and/or with anatomical image data relating to the examination object, which is captured by means of the imaging device, and
wherein the output of the item of intervention information includes outputting the intervention plan.

An intervention plan of the medical instrument can comprise, e.g., a navigation plan, in particular a planned path, a movement plan, and/or a movement trajectory of the medical instrument in an interior, in particular a body, of the examination object. The intervention plan can also contain an item of information relating to an incision point and/or a destination position of the medical instrument in the examination object. An intervention plan can comprise, in particular, an item of instrument information according to an aspect variant described above. It is equally conceivable for the intervention plan to comprise a planned effect of the medical instrument in the tissue of the examination object, e.g., a representation and/or an item of information relating to an ablation volume, a temperature distribution, a force distribution, or similar.

The registration of the intervention plan with the three-dimensional model of the examination object and/or with the anatomical image data relating to the examination object can take place according to an aspect variant described above.

By virtue of registering the intervention plan with the three-dimensional model of the examination object and/or with the anatomical image data relating to the examination object, an accuracy of an output of the item of intervention information onto the examination object can advantageously be increased.

In an aspect variant of the inventive method, the capture of the item of instrument information comprises capturing a position and/or orientation of a medical instrument in the examination object as a function of the anatomical image data relating to the examination object.

The capture of the position and/or orientation of the medical instrument in the examination object as a function of the image data relating to the examination object is preferably effected by means of an image processing algorithm. For example, the capture of the position and/or orientation of the medical instrument in the examination object can include segmenting the anatomical image data relating to the examination object. In this way, it is possible to identify not only anatomical structures but also the medical instruments and to determine their relative positions. It is equally conceivable for the capture of the position and/or orientation of the medical instrument to include identifying one or a plurality of markers of the medical examination object. For this purpose, the markers can be embodied in such a way that they are visible or distinguishable for a receiving system of the imaging device. For example, the markers can generate characteristic contrasts in magnetic resonance image data or computed tomography image data.

According to the disclosure, the provision of the item of intervention information includes providing a representation of the medical instrument, the output of the item of intervention information including outputting the representation of the medical instrument onto the examination object.

The capture of the position and/or orientation of the medical instrument in the examination object and the output of the representation of the medical instrument onto the examination object preferably take place essentially in real time. This can mean that a doctor performing treatment is able to detect a change in an orientation and/or position of the medical instrument with a small time delay of less than two seconds, less than one second, or preferably less than half a second as a function of the representation of the medical instrument that is output.

By means of the inventive method, a change in a position and/or orientation of the medical instrument can be provided in the image recording region, in particular on a surface of the examination object. It is advantageously possible thereby to avoid not only the need for the doctor performing treatment to look up repeatedly but also the time that this entails.

In a preferred aspect variant of the inventive method, the intervention plan comprises a navigation plan for the medical instrument in the examination object, the capture of the item of instrument information, including determining a degree of correspondence between the navigation plan for the medical instrument and the position and/or orientation of the medical instrument.

The determination of a degree of correspondence between the navigation plan for the medical instrument and the position and/or orientation of the medical instrument can take place as a function of segmented image data relating to the imaging device, for example. In particular, the determination of the degree of correspondence between the navigation plan for the medical instrument and the position and/or orientation of the medical instrument can include ascertaining a deviation between a desired position and/or orientation of the medical instrument according to a navigation plan and an actual position and/or orientation of the medical instrument at a given point in time (or essentially in real time).

According to the disclosure, the output of the item of intervention information includes outputting an item of information relating to the degree of correspondence between the navigation plan for the medical instrument and the position and/or orientation of the medical instrument in the examination object.

For example, the output of the item of intervention information can include outputting a desired destination position and/or a desired position and/or orientation of the medical instrument at a desired point in time of an interventional procedure. A current position and a desired position and/or destination position of the medical instrument can be provided as, e.g. a representations of the medical instrument, in particular, superimposed on each other. It is equally conceivable for the output of the item of intervention information to include outputting a correction instruction. A correction instruction can be any item of information that allows the doctor performing treatment to minimize a deviation between an actual position and/or orientation of the medical instrument and a desired position and/or orientation of the medical instrument at a given point in time. The correction instruction can be provided as, for example, a visual item of information, e.g., a representation of the medical instrument and/or a direction indicator, but also as a haptic item of information and/or an acoustic item of information, e.g., a vibration of a positioning unit and/or a voice instruction.

By virtue of outputting an item of information relating to the degree of correspondence between the navigation plan for the medical instrument and an actual position and/or orientation of the medical instrument in the examination object, any risk of incorrect positioning of the medical instrument can be advantageously reduced.

The inventive imaging device is designed to carry out a method according to an aspect variant described above. In particular, the inventive imaging device comprises all components required to carry out the described aspect variants of the inventive method. The inventive imaging device comprises a sensor, an output unit, and a computing unit.

The imaging device is designed to capture anatomical image data relating to an examination object. The imaging device can be embodied according to an aspect variant described above. In a preferred aspect variant, the imaging device is embodied as a magnetic resonance device.

According to the disclosure, the sensor is designed to capture an item of depth information relating to the examination object in an image recording region of the imaging device. The sensor can be embodied according to an aspect variant described above. It is conceivable for the sensor to be a component of the imaging device and/or structurally integrated into the imaging device. However, the sensor can also be embodied as a separate or independent component. The sensor preferably has a signal connection to the computing unit and/or a control unit of the imaging device in order to transfer the item of depth information, e.g., optical image data, to the computing unit and/or the control unit of the imaging device. In preferred aspect variants, the sensor is a camera and/or part of a receiving system, in particular an image data capture system, of the imaging device.

According to the disclosure, the computing unit is designed to provide an item of intervention information as a function of the item of depth information. The computing unit preferably comprises an image processing unit which is designed to process items of depth information and/or anatomical image data. The image processing unit can be designed in particular to segment structures in the items of depth information and/or in the anatomical image data and/or to determine an examination position of the examination object as a function of the items of depth information. The image processing unit can be further designed to register anatomical image data, items of depth information, and/or an item of intervention information with each other, according to an aspect variant described above. The image processing unit can comprise a plurality of subunits having various image processing functions.

The computing unit and/or a control unit of the imaging device preferably have an interface that is coupled to the sensor by means of a signal connection in order to capture items of depth information from the sensor.

According to the disclosure, the output unit is designed to output the item of intervention information into the image recording region. The output unit preferably comprises a projector or a plurality of projectors. A projector can be embodied as a light projector, for example, in particular a laser projector. The projector can be designed to project an item of intervention information according to an aspect variant described above onto a surface in an image recording region of the imaging device.

In a preferred aspect variant, the output unit is designed to project the item of intervention information onto an inner surface of a patient tunnel or onto a surface of the examination object. The output unit is preferably positioned outside the image recording region in order to avoid any interaction with a physical measuring principle of the imaging device. It is, however, equally conceivable that the output unit is highly compatible with magnetic fields or ionizing radiation. In such cases, the output unit can also be positioned within the image recording region, in particular on a wall of the patient tunnel. The output unit can be integrated into the imaging device or form an independent component. The output unit is preferably connected by means of a signal connection to the control unit and/or the computing unit of the imaging device. The control unit and/or the computing unit can be designed to trigger the output unit to output the item of intervention information into the image recording region.

The output unit is preferably designed to compensate for or equalize a curvature of a surface in such a way that a substantially distortion-free projection of the item of intervention information is provided for a doctor performing treatment. In particular, the output unit can have a manually and/or automatically adaptable lens system, which allows the item of intervention information that is output to be adapted to various geometries and/or examination positions of the examination object. Furthermore, the lens system of the output unit can be specifically adapted to a geometry of a patient tunnel, an examination position of the examination object and/or a component of the imaging device.

In an aspect variant, the output unit has a curved display or a curved video screen. In this case, the output unit is preferably integrated into a wall of the patient tunnel.

The inventive imaging device is preferably designed to provide the item of intervention information automatically as a function of the item of depth information and output it into the image recording region. It is further conceivable for the imaging device to be designed to provide the item of intervention information automatically as a function of the anatomical image data, in particular in real-time.

By means of the inventive imaging device, it is advantageously possible to provide a reliable and repeatable output of an item of intervention information in an image recording region, in particular on a curved surface of an examination object. The inventive imaging device shares the advantages of the inventive method according to an aspect variant described above.

In an aspect variant, the inventive imaging device also has a movement detection unit, which is designed to determine a movement of the examination object.

The movement detection unit can comprise, e.g., a force sensor and/or a pressure sensor, which is positioned at the examination object in an appropriate position for the application and is designed to detect a movement of the examination object as a function of an expansion of a metrically sensitive part and/or a force effect on a metrically sensitive part of the movement detection unit. It is equally conceivable for the movement detection unit to comprise an optical sensor, radar sensor, ultrasound sensor, ECG sensor, pilot tone sensor, or similar, which is designed to output a signal that is synchronized or correlated with a movement of the examination object (e.g., a movement of an extremity and/or a movement of an organ). In a preferred aspect variant, the movement detection unit is embodied as a respiration detection sensor. For example, the respiration detection sensor can comprise a belt, a strip, or a bandage that surrounds at least part of the thorax of a patient and is designed to extend as a result of a respiratory movement of the patient. The respiration detection sensor can further comprise a pressure sensor or an array of pressure sensors, which are integrated into a patient support device and designed to detect the movement of the examination object.

The movement detection unit can also be embodied as part of a receiving system, in particular, an image data capture system, of the imaging device. For example, the computing unit of the imaging device can be configured to detect a movement of the examination object as a function of captured anatomical image data relating to the examination object.

The movement detection unit is preferably connected by means of a signal connection to the control unit and/or the computing unit of the imaging device.

According to the disclosure, the computing unit is designed to provide the item of intervention information as a function of the detected movement of the examination object.

The computing unit can conceivably be designed to adapt the item of intervention information in order to compensate for and/or at least partially equalize movements of the examination object that have been identified. It is further conceivable for the computing unit to be designed to filter or select captured anatomical image data as a function of the movement that has been identified, in particular a respiration state of the examination object, and to provide the item of intervention information as a function of the filtered or selected anatomical image data. The item of intervention information can, therefore, be used to limit the output of anatomical image data to a specific respiratory state, for example. It is equally conceivable for the imaging device to be designed to limit the capture of anatomical image data as a function of a signal from the respiration detection sensor to a specific respiratory state.

As a result of providing an imaging device according to the disclosure with a respiration detection sensor, it is possible to avoid or reduce movement artifacts in anatomical image data relating to the examination object. It is thereby advantageously possible to improve a quality of the item of intervention information that is provided. Furthermore, the item of intervention information can be adapted to a movement of the examination object as a function of a signal from the movement detection unit, whereby an accuracy of the output of the item of intervention information in relation to the examination object can advantageously be improved.

The inventive computer program product can be loaded directly into a storage unit of a computing unit of an imaging device, according to the disclosure. The computer program product comprises program code means for performing an inventive method according to an aspect variant described above when the computer program product is executed in the computing unit of the imaging device.

By means of the inventive computer program product, the inventive method can be performed in a manner that is fast, identically repeatable, and resilient. The computer program product is configured in such a way that it can execute the inventive method steps by means of the computing unit. The computing unit must have relevant prerequisites in this case, e.g., a corresponding main memory, a corresponding graphics card, or a corresponding logic unit, so that the respective method steps can be executed efficiently. The computer program product is stored on a computer-readable medium, for example, on a network, a server, or a cloud, from where it can be loaded into a processor of the computing unit. In this case, the computing unit can be designed as an independent system component or as part of the imaging device. Furthermore, control information of the computer program product can be stored on an electronically readable data medium. The control information of the electronically readable data medium can be so configured as to perform a method according to the disclosure when the data medium is used in the computing unit of the imaging device. Examples of electronically readable data media are a DVD, magnetic tape, a USB stick, or any desired data storage entity on which electronically readable control information, in particular software, is stored. When this control information is read from the data medium and transferred to a control unit and/or the computing unit of the imaging device, all inventive aspect variants of the described inventive method can be performed.

### BRIEF DESCRIPTION OF THE DRAWINGS

Further advantages and details are revealed in the following description of exemplary aspects in connection with the drawings of schematic representations in which:

FIG. 1 shows a possible aspect variant of a magnetic resonance device according to the aspects of the disclosure,

FIG. 2 shows a possible aspect variant of a local coil according to the aspects of the disclosure,

FIG. 3 shows a possible aspect variant of a local coil according to the aspects of the disclosure and

FIG. 4 shows a possible aspect variant of a local coil according to the aspects of the disclosure.

### DETAILED DESCRIPTION

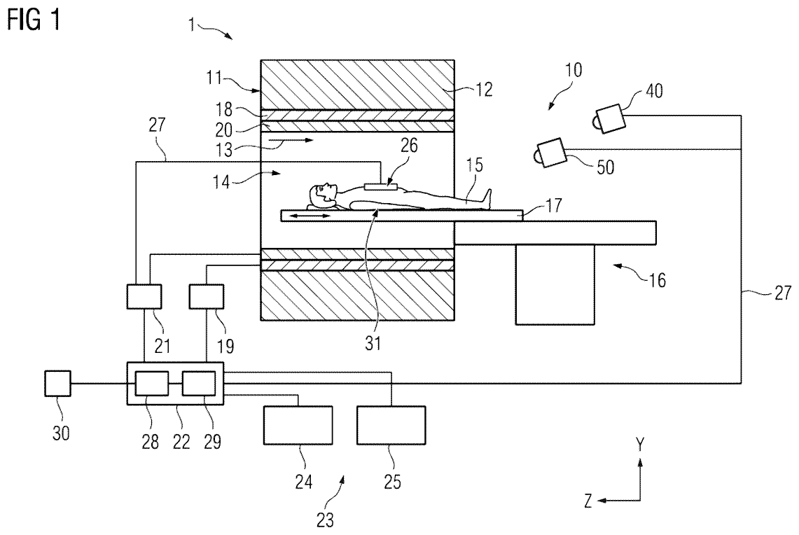

FIG. 1 shows an aspect variant of the inventive imaging device 1. The imaging device 1 here takes the form of a magnetic resonance device 10. The magnetic resonance device 10 comprises a magnet unit 11, which has, e.g., a permanent magnet, an electromagnet, or a superconducting main magnet 12 for generating a strong and, in particular, homogeneous main magnetic field 13 (BO magnetic field). The magnetic resonance device 10 also comprises a patient receiving region 14 for receiving a patient 15. The patient receiving region 14 in this exemplary aspect is of cylindrical design and is surrounded circumferentially by the magnet unit 11. However, designs of the patient receiving region 14, which vary from this example, are also conceivable. The patient receiving region 14 can correspond essentially to an image recording region of the imaging device 1.

In the example shown in FIG. 1, the examination object is a patient 15. The patient 15 can be positioned in the patient receiving region 14 by means of a patient support device 16 of the magnetic resonance device 10. For this purpose, the patient support device 16 has a patient table 17, which can be moved within the patient receiving region 14. The magnet unit 11 also has a gradient coil 18 for generating magnetic gradient fields, which are used for spatial encoding during a magnetic resonance measurement. The gradient coil 18 is activated by means of a gradient control unit 19 of the magnetic resonance device 10. The magnet unit 11 can also comprise a high-frequency antenna, this being designed in the present exemplary aspect as a body coil 20 which is permanently integrated into the magnetic resonance device 10. The body coil 20 is designed to excite nuclear spins, these being situated in the main magnetic field 13 which is generated by the main magnet 12. The body coil 20 is activated by a high-frequency unit 21 of the magnetic resonance device 10 and directs high-frequency excitation pulses into the image recording region, which is largely formed by a patient receiving region 14 of the magnetic resonance device 10. The body coil 20 is further designed to receive magnetic resonance signals and can represent a receiving unit or part of a receiving unit of the magnetic resonance device 10.

The magnetic resonance device 10 has a control unit 22 for controlling the main magnet 12, the gradient control unit 19 and the high-frequency unit 21. Control unit 22 is designed to control performance of an imaging sequence such as, e.g., a GRE (gradient echo) sequence, a TSE (turbo spin echo) sequence, or a UTE (ultra-short echo time) sequence. In addition, the control unit 22 comprises a computing unit 28 for evaluating magnetic resonance signals that are captured during a magnetic resonance measurement.

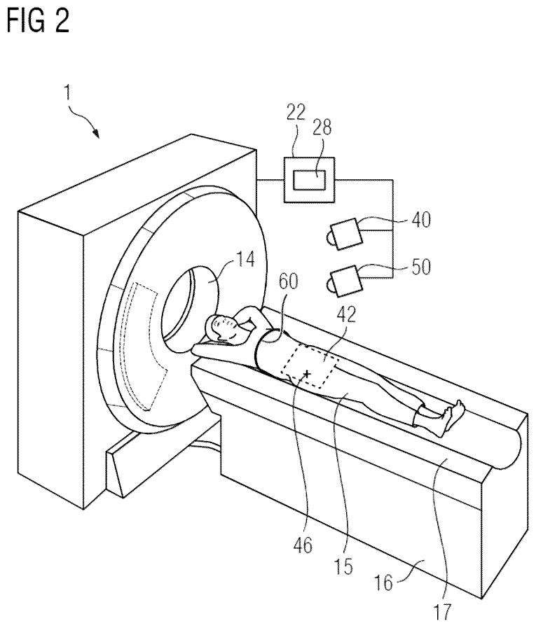

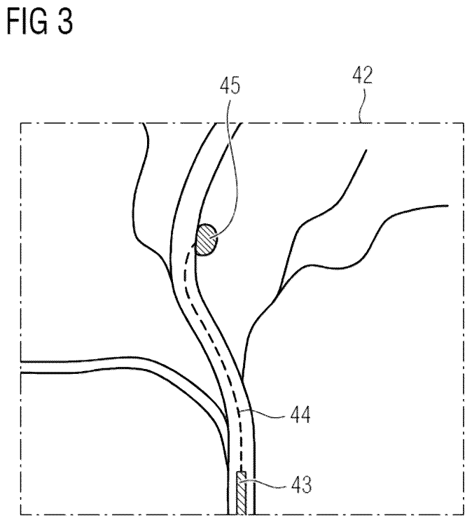

The control unit 22 and/or the computing unit 28 are preferably designed to trigger one or a plurality of sensors 40 to capture items of depth information from the patient 15. In this case, the patient 15 is preferably situated in the examination position. Furthermore, the control unit 22 and/or the computing unit 28 can be designed to trigger the output unit 50 to output an item of intervention information into the image recording region of the magnetic resonance device 10 (cf. FIGS. 2 and 3). The magnetic resonance device 10 can have one or more output units 50 and one or more sensors 40. In the present example, the sensor 40 is embodied as a three-dimensional camera. However, the sensor 40 can also represent part of a receiving system of the imaging device 1, e.g. a local coil 26 and/or a body coil 20 here.

The computing unit 28 of the magnetic resonance device 10 is preferably designed to capture and process items of depth information, anatomical image data, and/or a signal from a movement detection unit 60 by means of an interface and to provide an item of intervention information.

The magnetic resonance device 10 can comprise a user interface 23, which has a signal connection to the control unit 22. Control information, such as e.g. imaging parameters of the magnetic resonance measurement, can be shown on a display unit 24, e.g., at least one monitor of the user interface 23. The display unit 24 can be configured in particular to provide a graphical user interface with the representation of a relevant body region of the patient 15. The user interface 23 also has an input unit 25 by means of which parameters of a magnetic resonance measurement can be input by the user. The input unit can be configured in particular to allow a selection of one or a plurality of body regions by the user.

The computing unit 28 in the present example is connected to a storage unit 29 of the magnetic resonance device 10. Optionally, the computing unit 28 can also be connected to a cloud 30. The computing unit 28 can be configured to store data such as, e.g., items of depth information, anatomical image data, data from a movement detection unit 60, magnetic resonance images, x-ray images, or similar on the storage unit 29 and/or the cloud 30 and/or to retrieve data from the storage unit 29 and/or the cloud 30 by means of a suitable interface (not shown). The cloud 30 can conceivably be designed in particular to receive and process captured image data and/or images from the magnetic resonance device 10. For example, the Cloud 30 can be designed to perform a registration of items of depth information, anatomical image data, and/or a representation that is comprised of the item of intervention information and, on the basis of this, to provide an item of intervention information. The cloud 30 can also be designed to create a model of a patient as a function of captured items of depth information and/or anatomical image data. The cloud 30 can be designed in particular to transmit a result of the processing of the item of depth information and/or anatomical image data to the computing unit 28. The computing unit 28 can obviously also be designed to perform the tasks cited above.

The magnetic resonance device 10 can have further components such as, e.g., a local coil 26. The local coil 26 can be positioned at a diagnostically or therapeutically relevant body region of the patient 15 in an appropriate position for the application. The local coil 26 preferably has a plurality of antenna elements, which are designed to capture magnetic resonance signals from the relevant body region of the patient 15 and to transmit these to the computing unit 28 and/or the control unit 22. In order to achieve this, the local coil can be connected to the high-frequency unit 21 and the control unit 22 by means of an electrical interface cable 27 or other signal connection. Like the body coil 20, the local coil 26 can also be designed to excite nuclear spins in the jaw region 31 of the patient 15. The local coil 26 can be activated by the high-frequency unit 21 for this purpose. In an aspect variant, the local coil 26 is designed to capture items of depth information relating to the patient 15.

The sensor 40 can be embodied as a camera 40, e.g., a 2D camera, a 3D camera, an infrared camera, or similar. The sensor 40 is preferably designed to capture items of depth information, in particular optical data, optical image data, items of profile information and/or items of contour information relating to a patient 15 or to a surface of a patient 15. For the purpose of capturing the item of depth information, the patient 15 is preferably in the examination position in the patient receiving region 14 of the magnetic resonance device 10.

The output unit 50 here is embodied as a projector. The output unit 50 can, however, be embodied as a video screen that is positioned in the patient receiving region 14 of the imaging device 1 and/or is adapted to a geometry or curvature of a wall of a patient tunnel.

It is conceivable for the sensor 40 and/or the output unit 50 to be positioned outside the patient receiving region 14 or within the patient receiving region 14.

The illustrated imaging device 1 can obviously comprise further components that imaging devices usually have.

In FIG. 2, the inventive imaging device 1 is embodied as a computed tomography device. In the case of a computed tomography device, likewise, access to the patient 15 during an interventional procedure can be at least partly concealed and/or hampered by a patient tunnel that encloses the patient receiving region 14.

In the aspect variant shown, the imaging device 1 has a camera 40 for capturing optical data and a projector 50 for outputting an item of intervention information 42 onto a surface of the patient 15. During the capture of the optical data, the patient 15 is preferably positioned in the examination position in the patient receiving region 14. The camera 40 and/or the projector 50 can be manually and/or automatically adaptable to a position of the patient 15. In particular, the camera 40 and/or the projector 50 can be designed to follow a movement or positioning of the patient 15 by means of the patient table 17.

In the example shown, the imaging device 1 has a respiration detection sensor 60. The respiration detection sensor 60 here is embodied as a belt that surrounds at least a section of the thorax of the patient 15. When positioned on the patient 15 in an appropriate manner for the application, the belt is designed to expand as a result of inhalation and to output a signal that correlates to the expansion. The signal can be transmitted by means of a signal connection, e.g., an electrical signal cable or a wireless connection, to the computing unit 28 of the imaging device 1. The computing unit 28 can then provide the item of intervention information as a function of the signal from the respiration detection sensor 60. It is, however, equally conceivable for the computing unit 28 and/or the control unit 22 of the imaging device 1 to be designed to coordinate a capture of the anatomical image data of patient 15 as a function of the signal from the respiration detection sensor 60.

FIG. 2 schematically shows an item of intervention information 42, which is projected onto a surface of the patient 15 by means of the output unit 50. The patient 15 is preferably situated in an examination position in the patient receiving region 14 (not shown) when the item of intervention information is provided in the image recording region by means of the output unit 50. The output unit 50 can, however, also be designed to project the item of intervention information onto the patient when the latter is moved out of the patient receiving region 14 by means of the patient support device, as shown in FIG. 2.

FIG. 3 schematically shows an exemplary item of intervention information 42, which can be provided by the computing unit 28. The item of intervention information 42 here comprises anatomical image data relating to a view of the patient 15 with a blood vessel. The item of intervention information further comprises a representation of a catheter 43 and a navigation plan 44 and/or preferred movement trajectory of the catheter 43 through the blood vessel to a destination 45, this representing an aneurysm in the present example. In addition to the destination 45, the item of intervention information 42 can also comprise a representation of an incision point 46. The position and/or orientation of the representation of the medical instrument 43 can essentially be updated in real time. The representation of the medical instrument 43 can comprise a graphically simplified illustration of the medical instrument but can also comprise parts of the anatomical image data, in particular segmented extracts. It is further conceivable for the item of intervention information 42 to comprise an instruction for the correct orientation and/or positioning of the medical instrument. Such an instruction can include, e.g., a broken line that allows the medical instrument to be oriented and/or a representation of the medical instrument in a correct position and/or orientation. It is further conceivable for the item of intervention information 42 to contain a representation of a planned ablation volume and/or a planned temperature distribution (not shown). The item of intervention information 42 can also comprise an item of instrument information, e.g., an effect of the medical instrument 43 on a tissue of the examination object, in particular a current temperature distribution in the tissue of the examination object and/or a representation of a force effect of the medical instrument on the tissue of the examination object.

The item of instrument information 42, e.g., the representations shown in FIG. 3 of the catheter 43, the navigation plan 44, and/or the destination 45, can be registered with the anatomical image data by means of the computing unit 28 when the item of intervention information is provided. In addition to the destination 45, the item of intervention information 42 can also comprise a representation of an incision point 46 (see FIG. 2). Such a representation of the incision point 46 can comprise, e.g., a cross or a dot, which can be output onto the surface of the patient 15 in a correct position by means of the output unit 50.

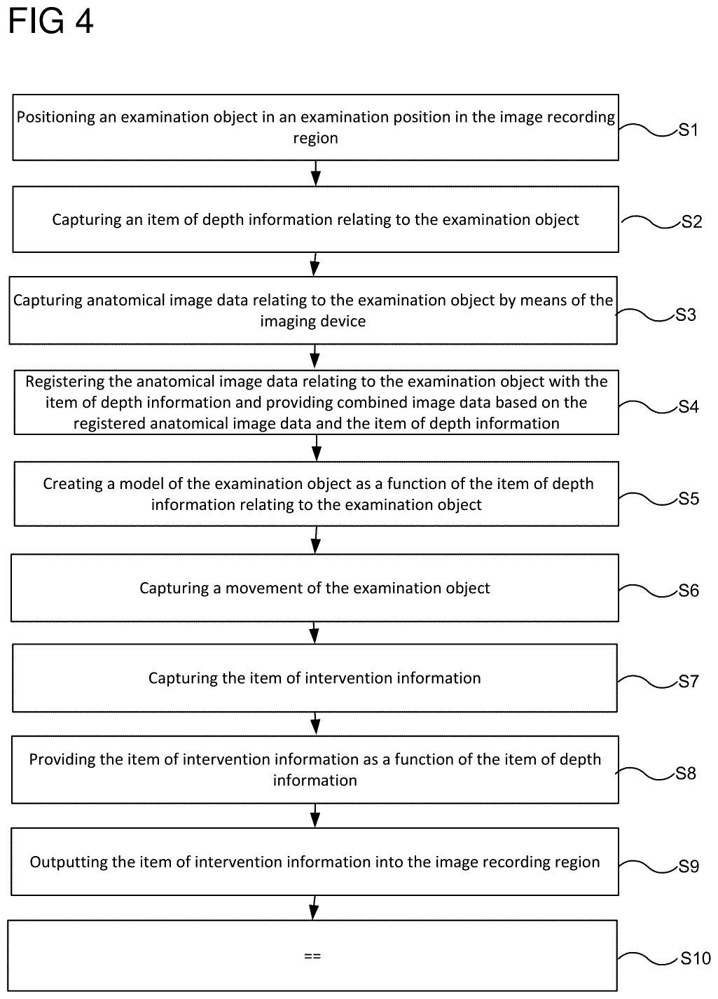

FIG. 4 schematically shows a sequence diagram of a method according to the disclosure. In the illustrated sequence diagram, the inventive method has the optional steps S3, S4, S5 and S6 (S3-S6). It is, however, conceivable to perform the inventive method without one or more of the steps S3-S6.

In the step S1, positioning of the examination object in an examination position takes place in the image recording region of the imaging device 1. During this activity, a patient 15 is preferably positioned on the patient table 17 in a posture required for the interventional procedure and/or the imaging examination, and is transported into the patient receiving region 14 of the imaging device 1 by means of the patient support device 16.

In a step S2, an item of depth information relating to the examination object is captured in an examination position in the image recording region. Optical data relating to the patient 15 is preferably captured by one or a plurality of cameras 40 as soon as the patient 15 occupies the examination position. The item of depth information is transmitted by means of a suitable signal connection 27 to the computing unit 28 and/or the control unit 22. It is conceivable for the computing unit 28 and/or the control unit 22 to be designed to trigger the camera 40 to capture items of depth information of the patient 15.

In the optional step S3, anatomical image data relating to the examination object is captured by means of the imaging device 1. The capture of the anatomical image data is preferably effected by means of a dedicated receiving system of the imaging device 1, e.g., by means of a body coil 20 and/or a local coil 26 of a magnetic resonance device 10, or by means of a detector system of a computed tomography device.

On the basis of the step S3, the anatomical image data relating to the examination object is registered with the item of depth information in the optional step S4 and combined image data is provided. For example, one of the image registration methods described above can be used for this purpose, in particular a feature-based method on the basis of a correlation function. The registration of the anatomical image data with the item of depth information is preferably effected by means of the computing unit 28 or a subunit of the computing unit 28. Provision of the combined image data can comprise, e.g., outputting by means of the output unit 50, transmitting the combined image data to a further subunit of the computing unit 28, and/or storing the combined image data on a local storage unit 29 and/or a storage unit of the cloud 30.

In an optional step, S5, a model of the examination object is created as a function of the item of depth information relating to the examination object. An algorithm (e.g., a neural network, in particular, a multilayer neural network) that has been trained on the basis of anatomical image data or a parameter-based model (e.g., based on dimensions or geometrical information from the optical data or items of depth information) is preferably used to provide a three-dimensional model of the patient 15. The creation of the model of the examination object is preferably effected by means of the computing unit 28, a subunit of the computing unit 28, and/or a computing unit in the cloud 30.

In a further optional step S6, a movement status of the examination object is captured. According to an example, the capture of the respiration state is effected by means of a respiration detection sensor 60, which is positioned on the patient 15 (see FIG. 2). The respiration detection sensor 60 can be embodied in particular as a belt that circumferentially surrounds a thorax of the patient 15 at least partly along a section. The respiration detection sensor 60 is preferably connected to the computing unit 28 by means of a suitable signal connection. The computing unit 28 can be designed accordingly to capture a signal from the respiration detection sensor 60. A signal from the respiration detection sensor can comprise any desired item of information, which contains an indication of a respiratory state of the patient 15.

In a step S7, an item of intervention information is captured. The capture of the item of intervention information preferably includes capturing an item of instrument information. The computing unit 28 can conceivably be designed to determine the item of intervention information automatically as a function of an item of patient information which is stored on the storage unit 29 and/or a signal connection with the medical instrument. Furthermore, the computing unit 28 can be designed to capture the item of intervention information as a function of a user input via the user interface 23. It is also conceivable for the computing unit 28 to obtain the item of intervention information as a function of a user input and/or an item of patient information from the cloud 30.

In the step S8, an item of intervention information is provided as a function of the item of depth information. The computing unit 28 is preferably designed to process the item of depth information and to provide the item of intervention information as a function of an item of information from the processed item of depth information.

In an aspect variant, the provision of the item of intervention information includes adapting the item of intervention information to the examination position of the patient 15. The computing unit 28 can be designed to determine a posture and/or a surface geometry of the patient 15 on the basis of the item of depth information, for example, and to adapt the item of intervention information in such a way that any distortion of an item of intervention information 42 which is projected onto the surface of the patient 15 can be avoided or reduced.

In the step S9, the item of intervention information (42) is output into the image recording region of the imaging device 1.

In further aspect variants of the inventive method, the provision of the item of intervention information can take place as a function of
the item of depth information and the anatomical image data relating to the examination object,
the combined image data,
the identified movement of the examination object and/or
the item of instrument information.

The computing unit 28 can be designed in particular to register a representation of the medical instrument 43, which is comprised in the item of intervention information, an incision point 46, a destination 45, and/or an intervention plan 44 of the medical instrument with the item of depth information, the anatomical image data and/or the combined image data. Furthermore, the computing unit 28 can be designed to adapt the item of intervention information as a function of a signal from a respiration detection sensor 60. Since the item of intervention information, e.g., a position of the incision point 46, a position of the destination 45, or a position and/or orientation of the medical instrument, can be associated with the coordinate system of the recorded anatomical image data, a registration of movement-dependent items of depth information and anatomical image data allows the items of intervention information to be transformed or adapted so that the output unit 50 can provide the items of intervention information on a surface of the patient 15.

In an aspect variant of the inventive method, the capture of the item of instrument information includes capturing an intervention plan. The computing unit 28 is preferably designed to determine the intervention plan as a function of an item of patient information and/or a user input.

The provision of the item of intervention information preferably includes registering the intervention plan with the model of the patient 15 and/or with the anatomical image data relating to the patient 15 by means of the computing unit 28. The intervention plan can then be output, as a result of the output unit 50 being triggered by the computing unit 28 and/or the control unit 22, onto a surface of the patient 15 or onto a surface in the image recording region of the imaging device 1.

In a further aspect variant of the inventive method, the capture of the item of instrument information includes capturing a position and/or orientation of the medical instrument in the patient 15 as a function of the anatomical image data of the patient 15. The computing unit 28 preferably has an image processing unit, which is designed to determine the position and/or orientation of the medical instrument in the patient 15 on the basis of the anatomical image data. The determination of the position and/or orientation of the medical instrument can be effected in particular by means of segmentation or a similar image processing method.

The computing unit 28 provides a representation of the medical instrument. The output of the representation of the medical instrument onto the surface of the patient 15 preferably takes place as a result of the output unit 50 being triggered by the computing unit 28 and/or the control unit 22.

In a further aspect variant of the inventive method, the intervention plan comprises a navigation plan for the medical instrument in the patient 15. The provision of the item of intervention information includes determining a degree of correspondence between the navigation plan for the medical instrument and the position and/or orientation of the medical instrument as a function of the anatomical image data relating to the patient 15. The determination of the degree of correspondence between the navigation plan for the medical instrument and the position and/or orientation of the medical instrument is preferably effected by means of segmentation, a trained image processing algorithm (e.g., an artificial neural network), edge recognition, a model of the medical instrument or/or a comparable image processing method.

In a preferred aspect variant, the computing unit 28 provides an item of information relating to the degree of correspondence between the navigation plan for the medical instrument and the actual position and/or orientation of the medical instrument in the patient 15. The item of information relating to the degree of correspondence between the navigation plan for the medical instrument and the position and/or orientation of the medical instrument can comprise, e.g., a graphical representation of the medical instrument in a desired position along a movement trajectory of the navigation plan, a correction instruction and/or any desired symbolic, graphical, numerical or textual indicator.

The output of the item of information relating to the degree of correspondence between the navigation plan for the medical instrument and the position and/or orientation of the medical instrument preferably takes place as a result of the output unit 50 being triggered by the computing unit 28 and/or the control unit 22.

Although the aspects of the disclosure are illustrated and described above by means of the preferred exemplary aspects, the aspects of the disclosure are not restricted by the examples disclosed, and other variations may be derived therefrom by a person skilled in the art without departing from the scope of the aspects of the disclosure.

In particular, the sequence of the method steps of the inventive method must be understood to be exemplary. The individual steps can also be performed in a sequence other than the sequence described or can coincide temporally, either in part or completely.

1. A computer-implemented method for providing an item of intervention information in an image recording region of an imaging device, the computer-implemented method comprising:
positioning an examination object in an examination position in the image recording region;
capturing an item of depth information relating to the examination object;
capturing the item of intervention information;
providing the item of intervention information as a function of the item of depth information and
outputting the item of intervention information into the image recording region.

2. The method as claimed in claim 1, wherein the output of the item of intervention information into the image recording region takes place on the examination object and/or a wall of the imaging device.

3. The method as claimed in claim 1, wherein the provision of the item of intervention information includes adapting the item of intervention information to the examination position of the examination object.

4. The method as claimed in claim 1, further comprising:
capturing anatomical image data relating to the examination object using the imaging device,
wherein the provision of the item of intervention information takes place as a function of the item of depth information and the anatomical image data relating to the examination object.

5. The method as claimed in claim 4, further comprising:
registering the anatomical image data relating to the examination object with the item of depth information and providing combined image data based on the registered anatomical image data and the item of depth information,
wherein the provision of the item of intervention information takes place as a function of the combined image data.

6. The method as claimed in claim 4, further comprising:
creating a model of the examination object as a function of the item of depth information relating to the examination object,
wherein the provision of the item of intervention information includes registering the model with the item of depth information, the anatomical image data and/or combined image data, and
wherein the combined image data is based on a registration of the anatomical image data relating to the examination object with the item of depth information.

7. The method as claimed in claim 1, further comprising:
capturing a movement of the examination object,
wherein the provision of the item of intervention information takes place as a function of the movement.

8. The method as claimed in claim 4,
wherein the capture of the item of intervention information includes capturing an item of instrument information,
wherein the provision of the item of intervention information takes place as a function of the item of instrument information and
wherein the output of the item of intervention information includes outputting the item of instrument information onto the examination object in the image recording region.

9. The method as claimed in claim 8,
wherein the capture of the item of instrument information includes capturing an intervention plan,
wherein the provision of the item of intervention information includes registering the intervention plan with a model that is created as a function of the item of depth information relating to the examination object and/or with anatomical image data relating to the examination object, which is captured using the imaging device, and
wherein the output of the item of intervention information includes outputting the intervention plan.

10. The method as claimed in claim 8,
wherein the capture of the item of instrument information includes capturing a position and/or orientation of a medical instrument in the examination object as a function of the anatomical image data relating to the examination object,
wherein the provision of the item of intervention information includes providing a representation of the medical instrument, and
wherein the output of the item of intervention information includes outputting the representation of the medical instrument onto the examination object.

11. The method as claimed in claim 9,
wherein the intervention plan comprises a navigation plan for the medical instrument in the examination object,
wherein the capture of the item of instrument information includes determining a degree of correspondence between the navigation plan for the medical instrument and the position and/or orientation of the medical instrument, and
wherein the output of the item of intervention information includes outputting an item of information relating to the degree of correspondence between the navigation plan for the medical instrument and the position and/or orientation of the medical instrument in the examination object.

12. The method as claimed in claim 1, wherein the item of intervention information is an element selected from a group of elements consisting of: an intervention plan, a navigation plan for a medical instrument in the examination object, an item of instrument information, a representation of a medical instrument, a representation of an incision point, a representation of an anatomical structure, a representation of a destination position, a representation of an ablation volume, a representation of a temperature distribution, a representation of a navigation plan and/or a representation of a degree of correspondence between a navigation plan for a medical instrument and a position and/or orientation of the medical instrument in the examination object.

13. An imaging device that is designed to execute the method as claimed in claim 1 and comprises:
a sensor
an output unit; and
a computing unit,
wherein the imaging device is designed to capture anatomical image data relating to an examination object,
wherein the sensor is designed to capture an item of depth information relating to the examination object in an image recording region of the imaging device,
wherein the computing unit is designed to provide an item of intervention information as a function of the item of depth information, and
wherein the output unit is designed to output the item of intervention information into the image recording region.

14. The imaging device as claimed in claim 13, further comprising:
a movement detection unit that is designed to identify a movement of the examination object,
wherein the computing unit is designed to provide the item of intervention information as a function of the identified movement of the examination object.

15. A non-transitory computer program product that is loadable directly into a data storage entity of a computing unit of an imaging device, comprising program code for executing a method as claimed in claim 1 when the non-transitory computer program product is executed in the computing unit of the imaging device.
